(19) United States
(12) Patent Application Publication
(10) Pub. No.: US 2023/0172965 A1
(43) Pub. Date: Jun. 8, 2023

(54) TARGETING LTBP3 FOR NONALCOHOLIC STEATOHEPATITIS (NASH)-INDUCED LIVER FIBROSIS

(71) Applicant: The Trustees of Columbia University in the City of New York, New York, NY (US)

(72) Inventors: Utpal Pajvani, Leonia, NJ (US); Brent Mayfield, New York, NY (US)

(73) Assignee: The Trustees of Columbia University in the City of New York, New York, NY (US)

(21) Appl. No.: 18/154,702

(22) Filed: Jan. 13, 2023

Related U.S. Application Data
(63) Continuation of application No. PCT/US21/42026, filed on Jul. 16, 2021.
(60) Provisional application No. 63/052,879, filed on Jul. 16, 2020.

(51) Int. Cl. A61K 31/7105; A61P 1/16; A61P 29/00; C12N 15/86; A61K 45/06

(52) U.S. Cl. CPC A61K 31/7105; A61P 1/16; A61P 29/00; C12N 15/86; A61K 45/06; C12N 2750/14143; C12N 2750/14171

(57) ABSTRACT

This invention relates to the treatment of fatty liver disease. More specifically, embodiments of the invention provide a pharmaceutical composition comprising a pharmaceutical carrier and a compound that reduces LTBP3 activity in liver cells.

This application claims priority of U.S. Provisional Application No. 63/052,879, filed Jul. 16, 2020, the content of which is hereby incorporated by reference in its entirety.

This invention was made with government support under grant number DK119767 awarded by the National Institute of Health and the National Institute of Diabetes and Digestive and Kidney Diseases. The government has certain rights in the invention.

Throughout this application, various publications are referred to by first author and year of publication. Full citations of these references can be found following the Examples. The disclosures of these publications in their entireties are hereby incorporated by reference into this application in order to more fully describe the state of the art to which this invention pertains.

### BACKGROUND OF THE INVENTION

Obesity has reached epidemic status in the United States—the Centers for Disease Control has stated that more than ⅓ of American adults are obese and estimated the medical costs attributable to obesity at $147 billion in 2008, a number that is likely to be significantly higher today and in the future.

Obesity manifests as multiple pathologic states in the liver. Insulin resistance in adipocytes results in unrestrained lipolysis, with consequent excess free fatty acid flux to the liver (Savage and Semple 2010). In a parallel pathogenic process, excess adiposity leads to insulin resistance, which begets the fasting hyperglycemia of Type 2 diabetes (T2D) (Lin and Accili 2011).

Compensatory hyperinsulinemia drives hepatic de novo lipogenesis mediated by the nutrient-sensitive mechanistic target of rapamycin (mTOR) pathway (Li, Brown et al. 2010), and couples with an impaired ability to catabolize and export fatty acids (Bugianesi, Gastaldelli et al. 2005), results in excess hepatocyte triglyceride accumulation, which is known as non-alcoholic fatty liver disease (NAFLD).

Fatty liver disease is a condition in which fat builds up in your liver, of which there are two types: NAFLD and alcoholic fatty liver disease (ALD), also called alcoholic steatohepatitis. Despite NAFLD and ALD having similar pathological spectra, the epidemiological and clinical characteristics of these two diseases differ (Toshikuni et al. 2014). The fatty degeneration of liver cells occurs to a greater degree in NAFLD than in ALD (Toshikuni et al. 2014). In contrast, inflammatory cell infiltration is more pronounced in ALD than in NAFLD (Toshikuni et al. 2014).

As both the prevalence of obesity and the frequency of imaging studies increase, the clinical diagnosis of the excess hepatic fat that defines NAFLD is increasingly common (Bhala, Jouness et al. 2013; Dongiovanni, Anstee et al. 2013). NAFLD prevalence is increasing in parallel with increased obesity. In data from the National Health and Nutrition Examination Survey (NHANES), prevalence of nonalcoholic fatty liver disease (NAFLD) in the United States population has increased from 5.5% in 1988 to 11% in 2008 and is now the leading cause of chronic liver disease in the United States (Younossi, Stepanova et al. 2011).

NAFLD ranges in severity from simple (benign) steatosis, to hepatocellular damage and necroinflammatory changes which define non-alcoholic steatohepatitis (NASH). NASH is a pathological diagnosis made at liver biopsy. Regrettably, this progression of NAFLD, a potential “pre-disease” states with prevalence approaching 30% in some populations (Bhala, Jouness et al. 2013), to NASH, which predisposes to cirrhosis and need for liver transplantation, is unpredictable for any given patient (Loria, Adinolfi et al. 2010).

The transition between NAFLD and NASH has an inadequately defined molecular signature, and biomarkers proposed to be mechanistic determinants of the process (Hashimoto and Farrell 2009; Malik, Change et al. 2009). Recent work suggests a “multiple-hit” hypothesis, (Tilg 2010; Day et al. 1998; Nagwa et al. 2015; Tariq et al. 2014) where the first hit of fat accumulation sensitizes the liver to further injury, mediated by cross-talk between hepatocytes and other liver residents to accelerate a fairly benign process to one that has severe clinical consequence without approved pharmacologic therapy (Hasimoto 2009; Carpino 2013).

NASH, defined by hepatocyte damage with associated inflammation and fibrosis, predisposes to cirrhosis and hepatocellular cancer, and is the fastest-growing reason for liver transplantation. NASH has no approved pharmacotherapy—as the prevalence of obesity-related NASH continues to rise, and available livers for transplantation remain limiting, this unmet need grows more urgent.

Available livers for transplantation will not keep pace with the expected growth in NASH over the next few decades—novel pathways are sought to both further our understanding of the pathophysiology of NAFLD/NASH as well as provide potential new pharmaceutical targets to assist in our management of obesity-related morbidity and mortality. Thus, new therapies are needed.

Notch is a highly conserved family of proteins critical for cell fate decision-making, but less is known about Notch action in mature tissue. Notch activity is present at low levels in normal liver, increases markedly in livers from obese patients and diet-induced or genetic mouse models of obesity, but is highest in patients with NASH and shows significant positive correlations with plasma ALT and NAFLD Activity Score.

The Notch signaling pathway is critical for cell fate decision-making in development, but our published data (Zhu et al, Science Translational Medicine, 2018) prove that “reactivated” Notch signaling, specifically in hepatocytes, induces insulin resistance, and by means of crosstalk with local hepatic stellate cells (HSC) transforms simple steatosis to NASH-associated fibrosis, the major determinant of morbidity/mortality in NASH patients and the likely clinical endpoint of all potential therapeutics. Recent data (Yu et al) suggest the proximal hit to increased hepatocyte Notch activity is increased hepatocyte expression of the Notch ligand, Jagged1. Genetic (hepatocyte-specific Jagged1 loss-of-function mice) and proof-of-principle pharmacologic (using ASO, and GalNAc-modified siRNA) establish hepatocyte Jagged1 as causal to NASH-induced liver fibrosis in mouse models. Human studies show increased liver JAGGED1 expression across multiple cohorts. In sum, these data indicate that hepatocyte Jag1 is a strong therapeutic target for NASH-induced liver fibrosis.

Parallel RNA sequencing in models of endogenous and forced hepatocyte Notch activity has revealed several interesting candidate hormonal effectors, including two (Osteopontin and MCP1) that have generated pharmaceutical interest and intriguing data.

Notch activity is further described in U.S. Patent and Application Nos. 61/800,180, 14/814,407, 15/976,534, 62/031,090, 62/242,888, 15/768,701; and PCT Nos. PCT/US2014/026717 and PCT/US2016/057166; the entire contents of which are incorporated by reference.

The administration of two drugs to treat a given condition, such as fatty liver disease, raises a number of potential problems. In vivo interactions between two drugs are complex. The effects of any single drug are related to its absorption, distribution, and elimination. When two drugs are introduced into the body, each drug can affect the absorption, distribution, and elimination of the other and hence, alter the effects of the other. For instance, one drug may inhibit, activate or induce the production of enzymes involved in a metabolic route of elimination of the other drug (Guidance for Industry, 2006). In one example, combined administration of GA and interferon (IFN) has been experimentally shown to abrogate the clinical effectiveness of either therapy. (Brod 2000) In another experiment, it was reported that the addition of prednisone in combination therapy with IFN-β antagonized its up-regulator effect. Thus, when two drugs are administered to treat the same condition, it is unpredictable whether each will complement, have no effect on, or interfere with, the therapeutic activity of the other in a human subject.

Not only may the interaction between two drugs affect the intended therapeutic activity of each drug, but the interaction may increase the levels of toxic metabolites (Guidance for Industry, 2006). The interaction may also heighten or lessen the side effects of each drug.

Hence, upon administration of two drugs to treat a disease, it is unpredictable what change will occur in the negative side profile of each drug. In one example, the combination of natalizumab and interferon β-1a was observed to increase the risk of unanticipated side effects. (Vollmer, 2008; Rudick 2006; Kleinschmidt-DeMasters, 2005; Langer-Gould 2005).

Additionally, it is difficult to accurately predict when the effects of the interaction between the two drugs will become manifest. For example, metabolic interactions between drugs may become apparent upon the initial administration of the second drug, after the two have reached a steady-state concentration or upon discontinuation of one of the drugs. (Guidance for Industry, 2006).

There thus remains a need for safe and effective treatments for liver disease, including NASH-induced liver fibrosis.

### SUMMARY OF THE INVENTION

The present invention provides a method of treating a subject afflicted with fatty liver disease comprising administering to the subject in need thereof a pharmaceutical composition comprising a pharmaceutical carrier and a compound that reduces LTBP3 activity in liver cells in an amount effective to treat the subject.

### BRIEF DESCRIPTION OF THE DRAWINGS

FIG. 1 shows graphical representations of RNA sequencing that identifies LTBP3 as a novel hepatocyte Notch target from Example 1. Panel A shows a Volcano plot from RNAseq of Venus+ vs. Venus− hepatocytes sorted from NASH diet-fed Transgenic Notch Reporter (TNR) mice. Panel B shows a Venn diagram of RNA sequencing results from AAV8-TBG-GFP (GFP) and non-tumor (NT) and tumor (T) regions from AAV8-Tbg-Cre-transduced NICD floxed mice. Panel C shows overlapped hits from TNR and L-NICD tumor and non-tumor that were analyzed by DAVID, with validated genes corresponding to secreted proteins shown in Panel D.

FIG. 2 shows graphical representations of Notch-induced LTBP3 expression in NASH from Example 1. Panel A shows Latent TGFβ binding protein 3 (LTBP3) expression is increased in livers from NASH diet-fed WT mice. Panel B shows AAV8-TBG-Cre transduction of NICD floxed mice induces hepatocyte-specific recombination to generate L-NICD mice that show increased liver LTBP3 mRNA. Panel C shows AAV8-TBG-Cre transduction of NICD floxed mice induces hepatocyte-specific recombination to generate L-NCID mice that show increased LTBP3 liver protein levels. Panel D shows Ad-NICD-transduced WT mouse primary hepatocytes that show increased LTBP3 gene expression. In all of the above experiments, the canonical Notch target HES1 is used as a positive control.

FIG. 3 shows a graphical representation of Hepatic Stellate cells from Example 2. Section A shows that siRNA to LTBP3 reduced the ability of Ad-NICD CM to increase HSC activity markers (i.e. Col1a1). Section B shows that siRNA to LTBP3 reduced the ability of Ad-NICD CM increased HSC activity markers (i.e. aSMA). Section C shows that siRNA to LTBP3 reduced the ability of Ad-NICD CM increased HSC activity markers (i.e. Timp1).

FIG. 4. LTBP3 binds complement proteins in NASH liver. A. Volcano plot of significantly increased co-IP'd liver proteins from NASH diet-fed wildtype mice transduced with Ad-LTBP3-FLAG (as compared to Ad-GFP), then subjected to anti-FLAG IP and LC/MS-MS. Red indicates proteins involved in the complement cascade. B. Confirmation of LTBP3 binding to complement proteins C3 and CFH in hepatocytes transduced Ad-LTBP3-FLAG (or Ad-GFP), then subjected to anti-FLAG IP and Western blot.

FIG. 5. Reversal of fibrosis.
A. Reversal of NASH diet-induced fibrosis with AAV8-TBG-shLtbp3.
B. Reversal of NASH diet-induced fibrosis with GalNAc-siLtbp3.
C. Reversal of Notch-mediated fibrosis with GalNAc-siLtbp3.
D. Effects of forced hepatocyte LTBP3 expression on liver pathology.

### DETAILED DESCRIPTION OF THE INVENTION

Each embodiment disclosed herein is contemplated as being applicable to each of the other disclosed embodiments. Thus, all combinations of the various elements described herein are within the scope of the invention.

### Embodiments of the Invention

The present invention provides a method of treating a subject afflicted with fatty liver disease comprising administering to the subject in need thereof a pharmaceutical composition comprising a pharmaceutical carrier and a compound that reduces LTBP3 activity in liver cells in an amount effective to treat the subject.

In an embodiment of the present invention, LTBP3 is a therapeutic target.

In another embodiment, LTBP3 is a diagnostic and/or subtyping tool for liver disorders such as NASH, liver fibrosis and HCC.

The gene latent transforming growth factor beta binding protein 3 (LTBP3) regulates transforming growth factor beta (TGFβ), which plays a role in fibrogenesis of liver and other tissues. LTBP3 is upregulated in the livers of NASH animal models at both RNA and protein levels, and it correlates with Notch activity in hepatocytes.

In an embodiment, reducing LTBP3 activity comprises decreasing LTBP3 expression.

In an embodiment, the treatment includes reducing the subject's hepatic triglyceride levels and collagen content.

In an embodiment the pharmaceutical composition decreases LTBP3 expression, thereby decreasing LTBP3 in the liver cells.

In an embodiment the pharmaceutical composition inhibits interactions of LTBP3 and pro-TGFβ homodimers, thereby decreasing LTBP3 in the liver cells.

In an embodiment the pharmaceutical composition inhibits interactions of LTBP3 and Rbp-Jk binding sites, thereby decreasing LTBP3 in the liver cells.

In an embodiment the fatty liver disease is nonalcoholic fatty liver disease or nonalcoholic steatohepatitis.

In an embodiment the pharmaceutical composition comprises anti-LTBP antibodies.

In an embodiment the pharmaceutical composition is targeted to the liver of the subject.

In an embodiment the administration of the LTBP3 inhibitor inhibits liver LTBP3 without significantly inhibiting LTBP elsewhere in the subject.

In an embodiment the LTBP3 inhibitor is a small molecule inhibitor, an oligonucleotide or an adenoviral vector.

In some embodiments, the oligonucleotide is an antisense oligonucleotide, an RNA-interference inducing compound, or a ribozyme.

In some embodiments, each compound administered to the subject is, independently, an organic compound having a molecular weight less than 1000 Daltons, a DNA aptamer, an RNA aptamer, a polypeptide, an antibody, an oligonucleotide, an interfering RNA (RNAi) molecule, a ribozyme, or a small molecule inhibitor.

In some embodiments, the oligonucleotide is targeted to hepatocytes.

In an embodiment the oligonucleotide is targeted to hepatocytes.

In an embodiment the oligonucleotide is modified to increase its stability in vivo.

In an embodiment the adenoviral vector is adenoviral shRNA. In an embodiment the pharmaceutical composition is administered in combination with Notch-active therapies.

In an embodiment the Notch-active therapy comprises a Notch1 decoy protein.

In an embodiment the Notch1 decoy protein comprises (a) amino acids, the sequence of which is identical to the sequence of a portion of the extracellular domain of a human Notch1 receptor protein and (b) amino acids, the sequence of which is identical to the sequence of an Fc portion of an antibody.

In an embodiment the Notch-active therapy comprises administering to the subject a Jagged inhibitor.

In an embodiment the Jagged inhibitor is small interfering RNA for JAG1.

Unless otherwise defined, all technical and scientific terms used herein have the same meaning as commonly understood by a person of ordinary skill in the art to which this invention belongs.

### Terms

As used in this application, except as otherwise expressly provided herein, each of the following terms shall have the meaning set forth below.

As used herein, the term “composition”, as in pharmaceutical composition, is intended to encompass a product comprising the active ingredient(s) and the inert ingredient(s) that make up the carrier, as well as any product which results, directly or indirectly from combination, complexation, or aggregation of any two or more of the ingredients, or from dissociation of one or more of the ingredients, or from other types of reactions or interactions of one or more of the ingredients.

As used herein, “effective amount” refers to an amount which is capable of treating a subject having a tumor, a disease or a disorder. Accordingly, the effective amount will vary with the subject being treated, as well as the condition to be treated. A person of ordinary skill in the art can perform routine titration experiments to determine such sufficient amount. The effective amount of a compound will vary depending on the subject and upon the particular route of administration used. Based upon the compound, the amount can be delivered continuously, such as by continuous pump, or at periodic intervals (for example, on one or more separate occasions). Desired time intervals of multiple amounts of a particular compound can be determined without undue experimentation by one skilled in the art. In one embodiment, the effective amount is between about 1 μg/kg-10 mg/kg. In another embodiment, the effective amount is between about 10 μg/kg-1 mg/kg. In a further embodiment, the effective amount is 100 μg/kg.

“Extracellular domain” as used in connection with Notch receptor protein means all or a portion of Notch which (i) exists extracellularly (i.e. exists neither as a transmembrane portion or an intracellular portion) and (ii) binds to extracellular ligands to which intact Notch receptor protein binds. The extracellular domain of Notch may optionally include a signal peptide (“sp”). “Extracellular domain”, “ECD” and “Ectodomain” are synonymous.

“Notch”, “Notch protein”, and “Notch receptor protein” are synonymous. In addition, the terms “Notch-based fusion protein” and “Notch decoy” are synonymous. The following Notch amino acid sequences are known and hereby incorporated by reference: Notch1 (Genbank accession no. S18188 (rat)); Notch2 (Genbank accession no. NP_077334 (rat)); Notch3 (Genbank accession no. Q61982 (mouse)); and Notch4 (Genbank accession no. T09059 (mouse)). The following Notch nucleic acid sequences are known and hereby incorporated by reference: Notch1 (Genbank accession no. XM_342392 (rat) and NM_017617 (human)); Notch2 (Genbank accession no. NM_024358 (rat), M99437 (human and AF308601 (human)); Notch3 (Genbank accession no. NM_008716 (mouse) and XM_009303 (human)); and Notch4 (Genbank accession no. NM_010929 (mouse) and NM_004557 (human)).

“Notch decoy protein”, as used herein, means a fusion protein comprising a portion of a Notch receptor protein which lacks intracellular signaling components and acts as a Notch signaling antagonist. Notch decoy proteins comprise all or a portion of a Notch extracellular domain including all or a portion of the EGF-like repeats present in the Notch extracellular domain. Examples of Notch decoy proteins include fusion proteins which comprise (a) amino acids, the sequence of which is identical to the sequence of a portion of the extracellular domain of a human Notch receptor protein and (b) amino acids, the sequence of which is identical to the sequence of an Fc portion of an antibody. In some Notch decoy proteins (b) is located to the carboxy terminal side of (a). Some Notch decoy proteins further comprise a linker sequence between (a) and (b). Notch decoy proteins can be selected from the group consisting of human Notch1 receptor protein, human Notch2 receptor protein, human Notch3 receptor protein and human Notch4 receptor protein. In some Notch decoy proteins the extracellular domain of the human Notch receptor protein is selected from the group consisting of Notch1 EGF-like repeats 1-36, Notch1 EGF-like repeats 1-13, Notch1 EGF-like repeats 1-24, Notch1 EGF-like repeats 9-23, Notch1 EGF-like repeats 10-24, Notch1 EGF-like repeats 9-36, Notch1 EGF-like repeats 10-36, Notch1 EGF-like repeats 14-36, Notch1 EGF-like repeats 13-24, Notch1 EGF-like repeats 14-24, Notch1 EGF-like repeats 25-36, Notch4 EGF-like repeats 1-29, Notch4 EGF-like repeats 1-13, Notch4 EGF-like repeats 1-23, Notch4 EGF-like repeats 9-23, Notch4 EGF-like repeats 9-29, Notch4 EGF-like repeats 13-23, and Notch4 EGF-like repeats 21-29.

Examples of Notch decoy proteins can be found in U.S. Pat. No. 7,662,919 B2, issued Feb. 16, 2010, U.S. Patent Application Publication No. US 2010-0273990 A1, U.S. Patent Application Publication No. US 2011-0008342 A1, U.S. Patent Application Publication No. US 2011-0223183 A1 and PCT International Application No. PCT/US2012/058662; the entire contents of each of which are hereby incorporated by reference into this application.

The terms “polypeptide,” “peptide” and “protein” are used interchangeably herein, and each means a polymer of amino acid residues. The amino acid residues can be naturally occurring or chemical analogues thereof. Polypeptides, peptides and proteins can also include modifications such as glycosylation, lipid attachment, sulfation, hydroxylation, and ADP-ribosylation.

“Subject” shall mean any organism including, without limitation, a mammal such as a mouse, a rat, a dog, a guinea pig, a ferret, a rabbit and a primate. In one embodiment, the subject is a human.

“Treating” means either slowing, stopping or reversing the progression of a disease or disorder. As used herein, “treating” also means the amelioration of symptoms associated with the disease or disorder.

As used herein, “agents for the treatment of fatty liver disease” are any agent known to or thought to treat a fatty liver disease. Agents for the treatment of obesity include, but are not limited to vitamin E, selenium, betadine, metformin, rosiglitazone, pioglitazone, insulin sensitizers, antioxidants, probiotics, Omega-3 DHA, pentoxifylline, anti-TNF-alpha, FXR agonists and GLP-1 agonists.

Units, prefixes and symbols may be denoted in their SI accepted form. Unless otherwise indicated, nucleic acid sequences are written left to right in 5′ to 3′ orientation and amino acid sequences are written left to right in amino- to carboxy-terminal orientation. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, “combination” means an assemblage of reagents for use in therapy either by simultaneous, contemporaneous, or fixed dose combination delivery. Simultaneous delivery refers to delivery of an admixture (whether a true mixture, a suspension, an emulsion or other physical combination) of the drugs. In this case, the combination may be the admixture or separate containers of the agents that are combined just prior to delivery. Contemporaneous delivery refers to the separate delivery of the agents at the same time, or at times sufficiently close together that an additive or preferably synergistic activity relative to the activity of either the agents is observed. Fixed dose combination delivery refers to the delivery of two or more drugs contained in a single dosage form for oral administration, such as a capsule or tablet.

### Inhibiting Expression of LTBP3

In some embodiments, the compound which is capable of inhibiting LTBP3 expression silences expression of a gene or silences transcription.

Oligonucleotides

Non-limiting examples of oligonucleotides capable of inhibiting LTBP3 expression include antisense oligonucleotides, ribozymes, and RNA interference molecules.

Antisense Oligonucleotides

Antisense oligonucleotides are nucleotide sequences which are complementary to a specific DNA or RNA sequence. Once introduced into a cell, the complementary nucleotides combine with natural sequences produced by the cell to form complexes and block either transcription or translation. Preferably, an antisense oligonucleotide is at least 11 nucleotides in length, but can be at least 12, 15, 20, 25, 30, 35, 40, 45, or 50 or more nucleotides long. Longer sequences also can be used. Antisense oligonucleotide molecules can be provided in a DNA construct and introduced into a cell as described above to decrease the level of target gene products in the cell.

Antisense oligonucleotides can be deoxyribonucleotides, ribonucleotides, or a combination of both. Oligonucleotides can be synthesized manually or by an automated synthesizer, by covalently linking the 5′ end of one nucleotide with the 3′ end of another nucleotide with non-phosphodiester internucleotide linkages such alkylphosphonates, phosphorothioates, phosphorodithioates, alkylphosphonothioates, alkylphosphonates, phosphoramidates, phosphate esters, carbamates, acetamidate, carboxymethyl esters, carbonates, and phosphate triesters.

Modifications of gene expression can be obtained by designing antisense oligonucleotides which will form duplexes to the control, 5′, or regulatory regions of the gene. Oligonucleotides derived from the transcription initiation site, e.g., between positions −10 and +10 from the start site, are preferred. Similarly, inhibition can be achieved using “triple helix” base-pairing methodology. Triple helix pairing is useful because it causes inhibition of the ability of the double helix to open sufficiently for the binding of polymerases, transcription factors, or chaperons. Therapeutic advances using triplex DNA have been described in the literature (Nicholls et al., 1993, J Immunol Meth 165:81-91). An antisense oligonucleotide also can be designed to block translation of mRNA by preventing the transcript from binding to ribosomes.

Precise complementarity is not required for successful complex formation between an antisense oligonucleotide and the complementary sequence of a target polynucleotide. Antisense oligonucleotides which comprise, for example, 1, 2, 3, 4, or 5 or more stretches of contiguous nucleotides which are precisely complementary to a target polynucleotide, each separated by a stretch of contiguous nucleotides which are not complementary to adjacent nucleotides, can provide sufficient targeting specificity for a target mRNA. Preferably, each stretch of complementary contiguous nucleotides is at least 4, 5, 6, 7, 8, 9, 10, 11, 12, 13, 14, 15, 16, 17, 18, 19, 20, 21, 22, 23, 24, 25, or more nucleotides in length. Noncomplementary intervening sequences are preferably 1, 2, 3, or 4 nucleotides in length. One skilled in the art can easily use the calculated melting point of an antisense-sense pair to determine the degree of mismatching which will be tolerated between a particular antisense oligonucleotide and a particular target polynucleotide sequence. Antisense oligonucleotides can be modified without affecting their ability to hybridize to a target polynucleotide. These modifications can be internal or at one or both ends of the antisense molecule. For example, internucleoside phosphate linkages can be modified by adding cholesteryl or diamine moieties with varying numbers of carbon residues between the amino groups and terminal ribose. Modified bases and/or sugars, such as arabinose instead of ribose, or a 3′, 5′-substituted oligonucleotide in which the 3′ hydroxyl group or the 5′ phosphate group are substituted, also can be employed in a modified antisense oligonucleotide. These modified oligonucleotides can be prepared by methods well known in the art.

Ribozymes

Ribozymes are RNA molecules with catalytic activity (Uhlmann et al., 1987, Tetrahedron. Lett. 215, 3539-3542). Ribozymes can be used to inhibit gene function by cleaving an RNA sequence, as is known in the art. The mechanism of ribozyme action involves sequence-specific hybridization of the ribozyme molecule to complementary target RNA, followed by endonucleolytic cleavage. Examples include engineered hammerhead motif ribozyme molecules that can specifically and efficiently catalyze endonucleolytic cleavage of specific nucleotide sequences. The coding sequence of a polynucleotide can be used to generate ribozymes which will specifically bind to mRNA transcribed from the polynucleotide. Methods of designing and constructing ribozymes which can cleave other RNA molecules in trans in a highly sequence specific manner have been developed and described in the art. For example, the cleavage activity of ribozymes can be targeted to specific RNAs by engineering a discrete “hybridization” region into the ribozyme. The hybridization region contains a sequence complementary to the target RNA and thus specifically hybridizes with the target RNA.

Specific ribozyme cleavage sites within an RNA target can be identified by scanning the target molecule for ribozyme cleavage sites which include the following sequences: GUA, GUU, and GUC. Once identified, short RNA sequences of between 15 and 20 ribonucleotides corresponding to the region of the target RNA containing the cleavage site can be evaluated for secondary structural features which may render the target inoperable. Suitability of candidate RNA targets also can be evaluated by testing accessibility to hybridization with complementary oligonucleotides using ribonuclease protection assays. Longer complementary sequences can be used to increase the affinity of the hybridization sequence for the target. The hybridizing and cleavage regions of the ribozyme can be integrally related such that upon hybridizing to the target RNA through the complementary regions, the catalytic region of the ribozyme can cleave the target.

Ribozymes can be introduced into cells as part of a DNA construct. Mechanical methods, such as microinjection, liposome-mediated transfection, electroporation, or calcium phosphate precipitation, can be used to introduce a ribozyme-containing DNA construct into cells in which it is desired to decrease target gene expression. Alternatively, if it is desired that the cells stably retain the DNA construct, the construct can be supplied on a plasmid and maintained as a separate element or integrated into the genome of the cells, as is known in the art. A ribozyme-encoding DNA construct can include transcriptional regulatory elements, such as a promoter element, an enhancer or VAS element, and a transcriptional teminator signal, for controlling transcription of ribozymes in the cells (U.S. Pat. No. 5,641,673). Ribozymes also can be engineered to provide an additional level of regulation, so that destruction of mRNA occurs only when both a ribozyme and a target gene are induced in the cells.

RNA Interference

An interfering RNA (RNAi) molecule involves mRNA degradation. The use of RNAi has been described in Fire et al., 1998, Carthew et al., 2001, and Elbashir et al., 2001, the contents of which are incorporated herein by reference.

Interfering RNA or small inhibitory RNA (RNAi) molecules include short interfering RNAs (siRNAs), repeat-associated siRNAs (rasiRNAs), and micro-RNAs (miRNAs) in all stages of processing, including shRNAs, pri-miRNAs, and pre-miRNAs. These molecules have different origins: siRNAs are processed from double-stranded precursors (dsRNAs) with two distinct strands of base-paired RNA; siRNAs that are derived from repetitive sequences in the genome are called rasiRNAs; miRNAs are derived from a single transcript that forms base-paired hairpins. Base pairing of siRNAs and miRNAs can be perfect (i.e., fully complementary) or imperfect, including bulges in the duplex region.

Interfering RNA molecules encoded by recombinase-dependent transgenes of the invention can be based on existing shRNA, siRNA, piwi-interacting RNA (piRNA), micro RNA (miRNA), double-stranded RNA (dsRNA), antisense RNA, or any other RNA species that can be cleaved inside a cell to form interfering RNAs, with compatible modifications described herein.

As used herein, an “shRNA molecule” includes a conventional stem-loop shRNA, which forms a precursor miRNA (pre-miRNA). “shRNA” also includes micro-RNA embedded shRNAs (miRNA-based shRNAs), wherein the guide strand and the passenger strand of the miRNA duplex are incorporated into an existing (or natural) miRNA or into a modified or synthetic (designed) miRNA. When transcribed, a shRNA may form a primary miRNA (pri-miRNA) or a structure very similar to a natural pri-miRNA. The pri-miRNA is subsequently processed by Drosha and its cofactors into pre-miRNA. Therefore, the term “shRNA” includes pri-miRNA (shRNA-mir) molecules and pre-miRNA molecules.

A “stem-loop structure” refers to a nucleic acid having a secondary structure that includes a region of nucleotides which are known or predicted to form a double strand or duplex (stem portion) that is linked on one side by a region of predominantly single-stranded nucleotides (loop portion). The terms “hairpin” and “fold-back” structures are also used herein to refer to stem-loop structures. Such structures are well known in the art and the term is used consistently with its known meaning in the art. As is known in the art, the secondary structure does not require exact base-pairing. Thus, the stem can include one or more base mismatches or bulges. Alternatively, the base-pairing can be exact, i.e. not include any mismatches.

“RNAi-expressing construct” or “RNAi construct” is a generic term that includes nucleic acid preparations designed to achieve an RNA interference effect. An RNAi-expressing construct comprises an RNAi molecule that can be cleaved in vivo to form an siRNA or a mature shRNA. For example, an RNAi construct is an expression vector capable of giving rise to a siRNA or a mature shRNA in vivo. Non-limiting examples of vectors that may be used in accordance with the present invention are described herein and will be well known to a person having ordinary skill in the art. Exemplary methods of making and delivering long or short RNAi constructs can be found, for example, in WO01/68836 and WO01/75164.

RNAi is a powerful tool for in vitro and in vivo studies of gene function in mammalian cells and for therapy in both human and veterinary contexts. Inhibition of a target gene is sequence-specific in that gene sequences corresponding to a portion of the RNAi sequence, and the target gene itself, are specifically targeted for genetic inhibition. Multiple mechanisms of utilizing RNAi in mammalian cells have been described. The first is cytoplasmic delivery of siRNA molecules, which are either chemically synthesized or generated by DICER-digestion of dsRNA. These siRNAs are introduced into cells using standard transfection methods. The siRNAs enter the RISC to silence target mRNA expression.

Another mechanism is nuclear delivery, via viral vectors, of gene expression cassettes expressing a short hairpin RNA (shRNA). The shRNA is modeled on micro interfering RNA (miRNA), an endogenous trigger of the RNAi pathway (Lu et al., 2005, Advances in Genetics 54: 117-142, Fewell et al., 2006, Drug Discovery Today 11: 975-982). Conventional shRNAs, which mimic pre-miRNA, are transcribed by RNA Polymerase II or III as single-stranded molecules that form stem-loop structures. Once produced, they exit the nucleus, are cleaved by DICER, and enter the RISC as siRNAs.

Another mechanism is identical to the second mechanism, except that the shRNA is modeled on primary miRNA (shRNAmir), rather than pre-miRNA transcripts (Fewell et al., 2006). An example is the miR-30 miRNA construct. The use of this transcript produces a more physiological shRNA that reduces toxic effects.

The shRNAmir is first cleaved to produce shRNA, and then cleaved again by DICER to produce siRNA. The siRNA is then incorporated into the RISC for target mRNA degradation. However, aspects of the present invention relate to RNAi molecules that do not require DICER cleavage. See, e.g., U.S. Pat. No. 8,273,871, the entire contents of which are incorporated herein by reference.

For mRNA degradation, translational repression, or deadenylation, mature miRNAs or siRNAs are loaded into the RNA Induced Silencing Complex (RISC) by the RISC-loading complex (RLC). Subsequently, the guide strand leads the RISC to cognate target mRNAs in a sequence-specific manner and the Slicer component of RISC hydrolyses the phosphodiester bound coupling the target mRNA nucleotides paired to nucleotide 10 and 11 of the RNA guide strand. Slicer forms together with distinct classes of small RNAs the RNAi effector complex, which is the core of RISC. Therefore, the “guide strand” is that portion of the double-stranded RNA that associates with RISC, as opposed to the “passenger strand,” which is not associated with RISC.

It is not necessary that there be perfect correspondence of the sequences, but the correspondence must be sufficient to enable the RNA to direct RNAi inhibition by cleavage or blocking expression of the target mRNA. In preferred RNA molecules, the number of nucleotides which is complementary to a target sequence is 16 to 29, 18 to 23, or 21-23, or 15, 16, 17, 18, 19, 20, 21, 22, 23, 24, or 25.

Isolated RNA molecules can mediate RNAi. That is, the isolated RNA molecules of the present invention mediate degradation or block expression of mRNA that is the transcriptional product of the gene. For convenience, such mRNA may also be referred to herein as mRNA to be degraded. The terms RNA, RNA molecule(s), RNA segment(s) and RNA fragment(s) may be used interchangeably to refer to RNA that mediates RNA interference. These terms include double-stranded RNA, small interfering RNA (siRNA), hairpin RNA, single-stranded RNA, isolated RNA (partially purified RNA, essentially pure RNA, synthetic RNA, recombinantly produced RNA), as well as altered RNA that differs from naturally occurring RNA by the addition, deletion, substitution and/or alteration of one or more nucleotides. Such alterations can include addition of non-nucleotide material, such as to the end(s) of the RNA or internally (at one or more nucleotides of the RNA). Nucleotides in the RNA molecules of the present invention can also comprise nonstandard nucleotides, including non-naturally occurring nucleotides or deoxyribonucleotides. Collectively, all such altered RNAi molecules are referred to as analogs or analogs of naturally-occurring RNA. RNA of the present invention need only be sufficiently similar to natural RNA that it has the ability to mediate RNAi.

As used herein the phrase “mediate RNAi” refers to and indicates the ability to distinguish which mRNA molecules are to be afflicted with the RNAi machinery or process. RNA that mediates RNAi interacts with the RNAi machinery such that it directs the machinery to degrade particular mRNAs or to otherwise reduce the expression of the target protein. In one embodiment, the present invention relates to RNA molecules that direct cleavage of specific mRNA to which their sequence corresponds. It is not necessary that there be perfect correspondence of the sequences, but the correspondence must be sufficient to enable the RNA to direct RNAi inhibition by cleavage or blocking expression of the target mRNA.

In some embodiments, an RNAi molecule of the invention is introduced into a mammalian cell in an amount sufficient to attenuate target gene expression in a sequence specific manner. The RNAi molecules of the invention can be introduced into the cell directly, or can be complexed with cationic lipids, packaged within liposomes, or otherwise delivered to the cell. In certain embodiments the RNAi molecule can be a synthetic RNAi molecule, including RNAi molecules incorporating modified nucleotides, such as those with chemical modifications to the 2′-OH group in the ribose sugar backbone, such as 2′-O-methyl (2′OMe), 2′-fluoro (2′F) substitutions, and those containing 2′OMe, or 2′F, or 2′-deoxy, or “locked nucleic acid” (LNA) modifications. In some embodiments, an RNAi molecule of the invention contains modified nucleotides that increase the stability or half-life of the RNAi molecule in vivo and/or in vitro. Alternatively, the RNAi molecule can comprise one or more aptamers, which interact(s) with a target of interest to form an aptamer:target complex. The aptamer can be at the 5′ or the 3′ end of the RNAi molecule. Aptamers can be developed through the SELEX screening process and chemically synthesized. An aptamer is generally chosen to preferentially bind to a target. Suitable targets include small organic molecules, polynucleotides, polypeptides, and proteins. Proteins can be cell surface proteins, extracellular proteins, membrane proteins, or serum proteins, such as albumin. Such target molecules may be internalized by a cell, thus effecting cellular uptake of the shRNA. Other potential targets include organelles, viruses, and cells.

As noted above, the RNA molecules of the present invention in general comprise an RNA portion and some additional portion, for example a deoxyribonucleotide portion. The total number of nucleotides in the RNA molecule is suitably less than in order to be effective mediators of RNAi. In preferred RNA molecules, the number of nucleotides is 16 to 29, more preferably 18 to 23, and most preferably 21-23.

Another tool for the integration of genes encoding peptides into the genome of a cell, is the clustered regularly interspaced short palindromic repeats (CRISPR)/Cas system, a system that originally evolved as an adaptive defense mechanism in bacteria and archaea against viral infection. The CRISPR/Cas system includes palindromic repeat sequences within plasmid DNA and an associated Cas9 nuclease. This ensemble of DNA and protein directs site specific DNA cleavage of a sequence of interest by first incorporating foreign DNA into CRISPR loci. Polynucleotides containing these foreign sequences and the repeat-spacer elements of the CRISPR locus are in turn transcribed in a host cell to create a guide RNA, which can subsequently anneal to a particular sequence and localize the Cas9 nuclease to this site. In this manner, highly site-specific cas9-mediated DNA cleavage can be engendered in a foreign polynucleotide because the interaction that brings cas9 within close proximity of the DNA molecule of interest is governed by RNA:DNA hybridization. As a result, one can theoretically design a CRISPR/Cas system to cleave any DNA molecule of interest. This technique has been exploited in order to edit eukaryotic genomes (Hwang et al., Nature Biotechnology 31:227 (2013)) and can be used as an efficient means of site-specifically editing cell genomes in order to cleave DNA prior to the incorporation of a gene encoding a gene. The use of CRISPR/Cas to modulate gene expression has been described in, for instance, U.S. Pat. No. 8,697,359, the disclosure of which is incorporated herein by reference as it pertains to the use of the CRISPR/Cas system for genome editing. Alternative methods for site-specifically cleaving genomic DNA prior to the incorporation of a gene of interest in a cell include the use of zinc finger nucleases (ZFNs) and transcription activator-like effector nucleases (TALENs). Unlike the CRISPR/Cas system, these enzymes do not contain a guiding polynucleotide to localize to a specific sequence. Sequence specificity is instead controlled by DNA binding domains within these enzymes. The use of ZFNs and TALENs in genome editing applications is described, e.g., in Urnov et al., Nature Reviews Genetics 11:636 (2010); and in Joung et al., Nature Reviews Molecular Cell Biology 14:49 (2013), the disclosure of each of which are incorporated herein by reference as they pertain to compositions and methods for genome editing.

Adenoviral Vector

An adenoviral vector encodes an oligonucleotide. The use of adenoviral vectors in gene therapy and tissue-specific targeting has been described in Beatty and Curiel, 2012, Barnett et al., 2002, and Rots et al., 2003, the contents of which are incorporated herein by reference.

### Methods of Administration

“Administering” compounds in embodiments of the invention can be effected or performed using any of the various methods and delivery systems known to those skilled in the art. The administering can be, for example, intravenous, oral, intramuscular, intravascular, intra-arterial, intracoronary, intramyocardial, intraperitoneal, and subcutaneous. Other non-limiting examples include topical administration, or coating of a device to be placed within the subject.

Injectable Drug Delivery

Injectable drug delivery systems may be employed in the methods described herein include solutions, suspensions, gels.

Oral Drug Delivery

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc). Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

For oral administration in liquid dosage form, an LTBP3 inhibitor may be combined with any oral, non-toxic, pharmaceutically acceptable inert carrier such as ethanol, glycerol, water, and the like.

Pharmaceutically Acceptable Carrier

The compounds used in embodiments of the present invention can be administered in a pharmaceutically acceptable carrier. As used herein, a “pharmaceutically acceptable carrier” is a pharmaceutically acceptable solvent, suspending agent or vehicle, for delivering the compounds to the subject. The carrier may be liquid or solid and is selected with the planned manner of administration in mind. Liposomes such as small unilamellar vesicles, large unilamallar vesicles, and multilamellar vesicles are also a pharmaceutically acceptable carrier. Liposomes can be formed from a variety of phospholipids, such as cholesterol, stearylamine, or phosphatidylcholines. The compounds may be administered as components of tissue-targeted emulsions. Examples of lipid carriers for antisense delivery are disclosed in U.S. Pat. Nos. 5,855,911 and 5,417,978, which are incorporated herein by reference. The compounds used in the methods of the present invention can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

A compound of the invention can be administered in a mixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone but are generally mixed with a pharmaceutically acceptable carrier. This carrier can be a solid or liquid, and the type of carrier is generally chosen based on the type of administration being used. In one embodiment the carrier can be a monoclonal antibody. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Specific examples of pharmaceutical acceptable carriers and excipients that may be used to formulate oral dosage forms of the present invention are described in U.S. Pat. No. 3,903,297, issued Sep. 2, 1975.

Tablets

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

Specific Administration To Liver

Embodiments of the invention relate to specific administration to the liver or hepatocytes.

In some embodiments, a compound may specifically target the liver.

In some embodiments, a compound may specifically target hepatocytes.

In some embodiments, a compound may be specifically targeted to the liver by coupling the compound to ligand molecules, targeting the compound to a receptor on a hepatic cell, or administering the compound by a bio-nanocapsule.

A compound of the invention can also be administered by coupling of ligand molecules, such as coupling or targeting moieties on preformed nanocarriers, such as (PGA-PLA nanoparticles, PLGA nanoparticles, cyclic RGD-doxorubicin-nanoparticles, and poly(ethylene glycol)-coated biodegradable nanoparticles), by the post-insertion method, by the Avidin-Biotin complex, or before nanocarriers formulation, or by targeting receptors present on various hepatic cell, such as Asialoglycoproein receptor (ASGP-R), HDL-R, LDL-R, IgA-R, Scavenger R, Transferrin R, and Insulin R, as described in: Mishra et al., (2013) Efficient Hepatic Delivery of Drugs: Novel Strategies and Their Significance, BioMed Research International 2013: 382184, dx.doi.org/10.1155/2013/382184, the entire contents of which are incorporated herein by reference.

A compound of the invention can also be administered by bio-nanocapsule, as described in: Yu et al., (2005) The Specific delivery of proteins to human liver cells by engineered bio-nanocapsules, FEBS Journal 272: 3651-3660, dx.doi.org/10.1111/j.1742-4658.2005.04790.x, the entire contents of which are incorporated herein by reference.

In some embodiments, an oligonucleotide specifically targets the liver.

In some embodiments, an oligonucleotide specifically targets hepatocytes.

Antisense oligonucleotides of the invention can also be targeted to hepatocytes, as described in: Prakash et al., (2014) Targeted delivery of antisense oligonucleotides to hepatocytes using triantennary N-acetyl galactosamine improves potency 10-fold in mice, Nucleic Acids Research 42(13): 8796-8807, dx.doi.org/10.1093/nar/gku531, the entire contents of which are incorporated herein by reference.

As used herein, the term “effective amount” refers to the quantity of a component that is sufficient to treat a subject without undue adverse side effects (such as toxicity, irritation, or allergic response) commensurate with a reasonable benefit/risk ratio when used in the manner of this invention, i.e. a therapeutically effective amount. The specific effective amount will vary with such factors as the particular condition being treated, the physical condition of the patient, the type of subject being treated, the duration of the treatment, the nature of concurrent therapy (if any), and the specific formulations employed and the structure of the compounds or its derivatives.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

The dosage of a compound of the invention administered in treatment will vary depending upon factors such as the pharmacodynamic characteristics of the compound and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds of the invention may comprise a compound alone, or mixtures of a compound with additional compounds used to treat cancer. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection or other methods, into the eye, all using dosage forms well known to those of ordinary skill in the pharmaceutical arts.

In an embodiment, the compound that decreases LTBP3 activity may be administered once a day, twice a day, every other day, once weekly, or twice weekly.

In an embodiment, 0.01 to 1000 mg of a compound that decreases LTBP3 activity is administered per administration.

Where a range is given in the specification it is understood that the range includes all integers and 0.1 units within that range, and any sub-range thereof. For example, a range of 1 to 5 is a disclosure of 1.0, 1.1, 1.2, etc.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### Example 1

There is significant rationale for combining Notch-active therapies (i.e. siJag1) with parallel therapeutic approaches against novel NASH targets. The targets here are based on a target identified as upregulated in hepatocytes derived from NASH diet-fed mice but are not direct Notch targets.

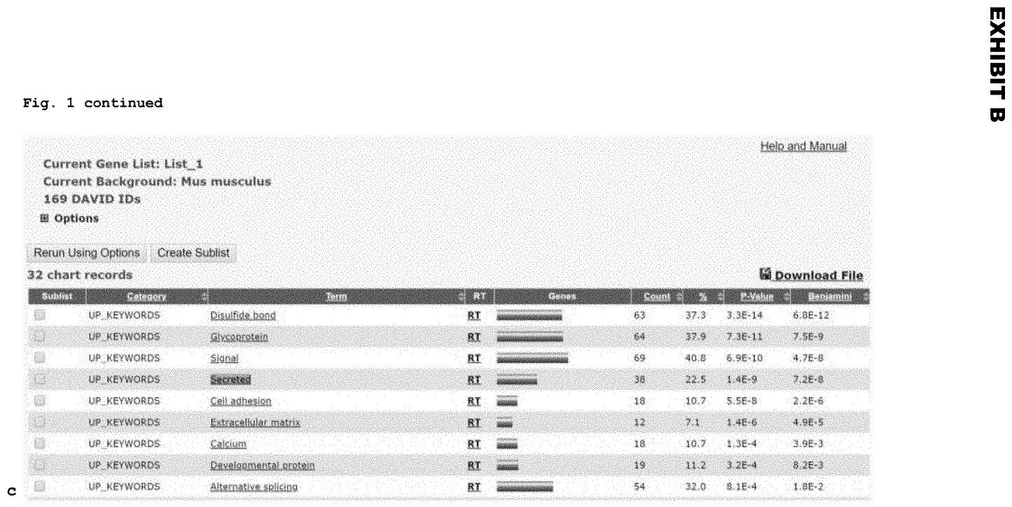

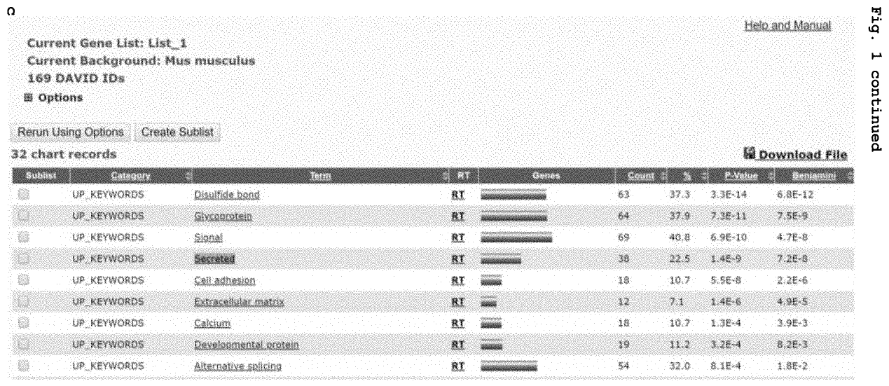

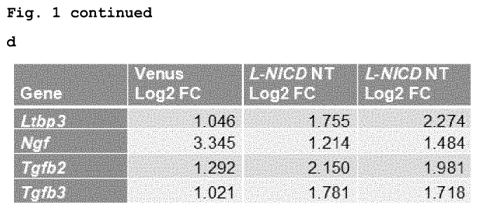

Hepatocyte-specific Notch gain-of-function (L-NICD) mice show increased liver fibrosis. Some of this is attributable to increased hepatocyte Osteopontin secretion. Osteopontin knockdown significantly reduces Notch-induced liver fibrosis but does not eliminate it. Compared to Ad-H1-shOpn transduced Cre-mice, Ad-H1-shOpn transduced L-NICD mice still have ˜2x the liver fibrosis (Zhu et al, 2018). To examine the additional Notch-dependent factors that influence liver collagen deposition, we perform parallel RNA sequencing analysis from models of endogenous (sorted Venus+ hepatocytes from Transgenic Notch Reporter mice fed a NASH-provoking diet) and exogenous Notch (L-NICD mice) activity. We overlay these data sets and examine significantly different and directionally consistent genes that overlap in both populations. We next use DAVID software to narrow to secreted factors that may be involved in hepatocyte-nonparenchymal (NPC) crosstalk to induce liver fibrosis (FIG. 1). This screen generates several known targets in NASH and fibrosis-Cc12 (i.e. monocyte chemoattractant protein 1) and Spp1 (Osteopontin)—an indication that Notch inhibition may be an excellent means to simultaneously block multiple pathogenic processes in liver. But this screen uncovers a novel target: LTBP3.

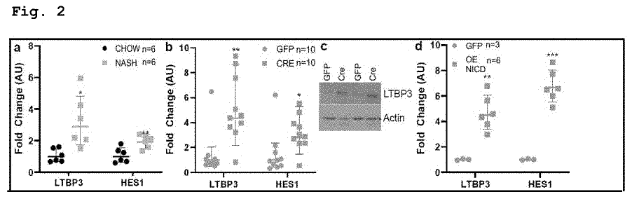

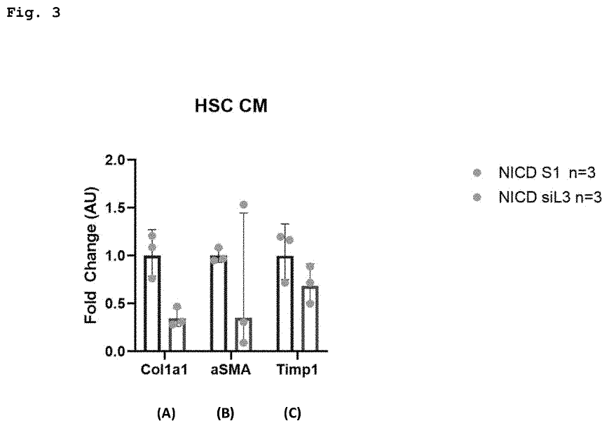

We observe increased LTBP3 expression in WT animals fed NASH diet, as well as in L-NICD mice and Ad-NICD-transduced mouse primary hepatocytes (FIG. 2A, 2B, 2C), that translates to greater hepatocyte LTBP3 protein levels (FIG. 2D).

### Example 2

We identified an evolutionarily conserved Rbp-Jk binding site in the LTBP3 promoter. Based on this data, we predict that Notch/Rbp-Jk transcriptionally activates hepatocyte LTBP3 expression.

Ad-NICD transduction of hepatocytes isolated from WT mice show increased LTBP3 expression. This data suggests that hepatocyte Notch activation may induce HSC activation through cell-autonomous increase of this factors. To test this hypothesis, we apply CM from Ad-NICD (or Ad-GFP control) transduced hepatocytes to HSCs isolated from WT mice. We observe that Ad-NICD CM increased HSC activity markers (i.e. Col1a1), suggesting that a Notch-stimulated secreted factor is at least partially responsible for HSC activation. But siRNA-mediated knockdown of hepatocyte LTBP3 reduces the potency of Ad-NICD CM to activate HSCs.

Next, to test whether LTBP3 mediates Notch-induced fibrosis in vivo, we will transduce adult, NASH diet-fed L-NICD mice with AAV8-H1-shLTBP3 or AAV8-H1-shScramble, which confers specificity to hepatocytes based on AAV8 trophism and the H1 promoter. We are cognizant that the shRNA approach may be insufficiently specific—thus in parallel, CRISPR is used as a parallel approach. We transduce Cre-dependent Cas9 knock-in mice (Jackson #026175) with AAV8-TBG-Cre:U6-LTBP3 sgRNA (or AAV8-TBG-Cre as a control) that we've generated to create hepatocyte-specific LTBP3 (L-LTBP3) knockout mice. We feed these mice NASH diet for 16 weeks, and test whether loss of either can ameliorate liver fibrosis. In addition, we have crossed Cas9 and NICD knock-in mice; when fed chow-diet, these mice mimic the increased liver fibrosis of L-NICD mice. Thus, with AAVs generated above, we can test whether LTPB3 specifically mediates Notch-induced fibrosis. It is expected that inhibition of hepatocyte proNGF or LTBP3 in L-NICD mice or hepatocytes will block Notch-induced HSC-activation and liver fibrosis.

These experiments test necessity, but in parallel, to test whether excess LTBP3 is sufficient to cause liver fibrosis, we generated AAV8 vectors for hepatocyte-specific LTBP3 overexpression (by means of the hepatocyte-specific promoter TBG), which we apply to both chow and NASH-diet fed mice. We expect a similar increase in liver fibrosis as seen in L-NICD mice. But as HSC activation may regulate hepatic injury, inflammatory tone and ductular reaction, we assess liver injury (ALT, TUNEL staining), ductal proliferation (CK19 staining), inflammation (CD45 staining), fibrosis (Sirius Red staining), and follow with qPCR studies to determine expression of markers of HSC activation.

### Example 3

We test the effect of GalNAc-siLTBP3 in mice with established fibrosis, in order to predict effects in patients most likely to be treated (based on biopsy-proven or radiologic imaging highly suggestive of established fibrosis). To do this, we perform the following experiments in C57/B16 wildtype mice:
a. 8 weeks NASH diet→8 weeks siLTBP3 (16 weeks total)—this experiment utilizes TD.190142 (Envigo) supplemented with glucose/sucrose-containing drinking water, which results in approximately 1% fibrosis at 16 week of diet feeding in C57 background (starting at 8 weeks of life), which goes up to 4-6% fibrosis at 32 weeks of diet.
b. 4 weeks HFD-CDAA diet→8 weeks siLTBP3 (12 weeks total)—in parallel, we use A06071302 (Research Diets) which induces similar fibrosis as TD.190142 at 12 weeks of diet feeding as 32 weeks of the Envigo diet, but with less weight gain/insulin resistance. This experiment ensures reproducibility of our findings.

### Example 4

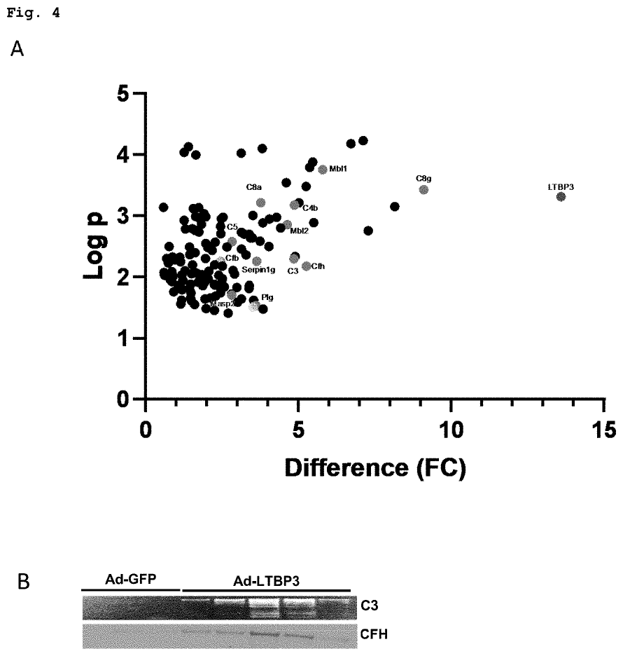

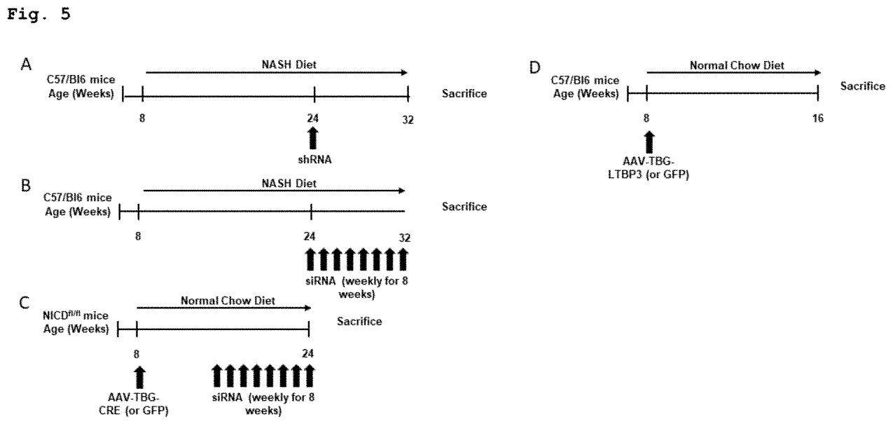

LTBP3 binding of complement proteins in NASH liver was analyzed. Volcano plot from mass spectrometry data indicates increased co-immunoprecipitation of proteins involved in the complement cascade in NASH diet-fed wildtype transduced with Ad-LTBP3-FLAG vs. Ad-GFP (Control) followed by LC/MS-MS (FIG. 4A). Western Blot confirms demonstrated interaction between LTBP3 and complement proteins C3 and CFH in hepatocytes transduced with AD-LTBPS vs. control (FIG. 4B).

### Example 5

“Reversal”-type experiments are conducted as shown in FIG. 5. These include reversal of NASH diet induced fibrosis with shLTBP3: 24 weeks NASH diet (C57/B16 wildtype mice) followed by shLTBP3 treatment and sacrifice at 32 weeks (FIG. 5A); We anticipate that reduction of Ltbp3 by AAV8-TBG-shLtbp3 transduction will reverse fibrosis in NASH diet-fed mice; reversal of NASH diet induced fibrosis with siLTBP3: 24 weeks NASH diet (C57/B16 wildtype mice)→8 weeks siLTBP3, weekly (32 weeks total) (FIG. 5B); We anticipate that reduction of Ltbp3 by weekly treatment with GalNAc-siLtbp3 will reverse fibrosis in NASH diet-fed mice; reversal of Notch-mediated fibrosis with siLTBP3: 24 weeks normal chow diet in AAV8-TBG-CRE (or GFP) transduced NICD floxed mice→8 weeks siLTBP3, weekly; (FIG. 5C) (24 weeks total); We anticipate that reduction of Ltbp3 by weekly treatment with GalNAc-siLtbp3 will reverse fibrosis in NASH diet-fed mice; effects of forced hepatocyte LTBP3 expression on liver pathology: 16 weeks normal chow diet in AAV-TBG-LTBP3 (or GFP) transduced C57/B16 wildtype mice (16 weeks total) (FIG. 5D); We anticipate that AAV8-TBG-Ltbp3 will cause liver fibrosis even in normal chow-fed mice.

As sex may be a biological variable, we perform these experiments in female mice.

### Example 6

Finally, to test “on-target” effects, to guide development of more effective therapeutics and predict potential toxicity, we administer siLTBP3 to L-DNMAML (hepatocyte-specific Notch loss-of-function) mice—if LTBP3-induced NASH/fibrosis is due to hepatocyte Notch receptors, we expect no additional benefit of lowering LTBP3 expression.

### Discussion

Notch—a receptor protein named for a characteristic wing defect in fruit flies (caused when the Notch gene is mutated)—mediates cell-fate decisions by coordinating interactions between neighboring cells expressing cognate receptors and ligands (Zhu et al. in press). In mammals, there are five surface-borne ligands of Notch proteins: Jagged (Jag1 and Jag2) and Delta-like (D111, D113, D114) families, each of which binds one of four Notch receptors (Notch 1-4) on a neighboring cell.

Ligand binding results in γ-secretase-mediated cleavage of the Notch receptor, which then sets off a pathway that culminates in the activation of a transcriptional regulator Hes1, which refines the Notch signal. Notch signaling is increased in NASH patients (Valenti et al. 2013), and specifically associated with the development of liver fibrosis (Zhu et al. 2018).

Latent transforming growth factor beta binding protein 3 (LTBP3) has been shown to regulate Transforming Growth Factor β TGFβ) action in cancer and other contexts (Robertson et al., 2015; Deryugina et al., 2018). While the latter's role in fibrogenesis in liver and other tissues is well-established, far less is known about pathophysiologic roles of LTBP3.

What is known, is that LTBPs (a family of 4 related peptides) binds a pro-TGFβ homodimer in the endoplasmic reticulum (ER), which facilitates cleavage of pro-TGFβ in the Golgi (Saharinen and Keski-Oja, 2000), and subsequent secretion as a large latent complex (LLC). Secreted LLC is anchored to the extracellular matrix and represents a releasable pool of “latent” TGFβ that can bind TGFβ receptor.

LTBP3 is the most abundantly expressed LTBP family member in liver, and the only one that strongly correlates with hepatocyte Notch activity.

### References

Savage, D. B. & Semple, R. K. Recent insights into fatty liver, metabolic dyslipidaemia and their links to insulin resistance. Current opinion in lipidology 21, 329-336 (2010).

Lin, H. V. & Accili, D. Hormonal regulation of hepatic glucose production in health and disease. Cell metabolism 14, 9-19 (2011).

Li, S., Brown, M. S. & Goldstein, J. L. Bifurcation of insulin signaling pathway in rat liver: mTORC1 required for stimulation of lipogenesis, but not inhibition of gluconeogenesis. Proceedings of the National Academy of Sciences of the United States of America 107, 3441-3446 (2010).

Bugianesi, E., et al. Insulin resistance in non-diabetic patients with non-alcoholic fatty liver disease: sites and mechanisms. Diabetologia 48, 634-642 (2005).

Toshikuni, N., et al. Clinical differences between alcoholic liver disease and nonalcoholic fatty liver disease. World J Gastroenterol. 20(26), 8393-8406 (2014).

Bhala, N., Jouness, R. I. & Bugianesi, E. Epidemiology and Natural History of Patients with NAFLD. Curr Pharm Des (2013).

Dongiovanni, P., Anstee, Q. M. & Valenti, L. Genetic Predisposition in NAFLD and NASH: Impact on Severity of Liver Disease and Response to Treatment. Curr Pharm Des (2013).

Younossi, Z. M., et al. Changes in the prevalence of the most common causes of chronic liver diseases in the United States from 1988 to 2008. Clin Gastroenterol Hepatol 9, 524-530 e521; quiz e560 (2011).

Loria, P. et al. Practice guidelines for the diagnosis and management of nonalcoholic fatty liver disease. A decalogue from the Italian Association for the Study of the Liver (AISF) Expert Committee. Dig Liver Dis 42, 272-282 (2010).

Hasimoto, E. & Farrell, G. C. Will non-invasive markers replace liver biopsy for diagnosing and staging fibrosis in non-alcoholic steatohepatitis? J Gastroenterol Hepatol 24, 501-503 (2009).

Malik, R., et al. The clinical utility of biomarkers and the nonalcoholic steatohepatitis CRN liver biopsy scoring system in patients with nonalcoholic fatty liver disease. J Gastroenterol Hepatol 24, 564-568 (2009).

Tilg, H. & Moschen, A. R. Evolution of inflammation in nonalcoholic fatty liver disease: the multiple parallel hits hypothesis. Hepatology 52, 1836-1846 (2010).

Day, C. P. & James, O. F. Steatohepatitis: a tale of two “hits”? Gastroenterology 114, 842-845 (1998).

Nakagawa, H. Recent advances in mouse models of obesity and nonalcoholic steatohepatitis-associated hepatocarcinogenesis. World journal of hepatology 7, 2110-2118 (2015).

Tariq, Z., Green, C. J. & Hodson, L. Are oxidative stress mechanisms the common denominator in the progression from hepatic steatosis towards non-alcoholic steatohepatitis (NASH)? Liver Int 34, e180-190 (2014).

Carpino, G., Renzi, A., Onori, P. & Gaudio, E. Role of hepatic progenitor cells in nonalcoholic fatty liver disease development: cellular cross-talks and molecular networks. Int J Mol Sci 14, 20112-20130 (2013).

Zhu et al, Science Translational Medicine, 2018.

Zhu et al, Nat Rev Gastro Hep, in press.

Valenti et al, Diabetes, 2013.

Robertson et al., Matrix Biology, 2015.

Deryugina et al, Oncogene, 2018.

Saharinen and Keski-Oja, Mol Biol Cell, 2000.

1. A method of treating a subject afflicted with fatty liver disease comprising administering to the subject in need thereof a pharmaceutical composition comprising a pharmaceutical carrier and a compound that reduces LTBP3 activity in liver cells in an amount effective to treat the subject.

2. The method of claim 1, wherein reducing LTBP3 activity comprises decreasing LTBP3 expression.

3. The method of claim 1, wherein the compound comprises a LTBP3 inhibitor and/or the compound decreases LTBP3 in liver cells.

4. (canceled)

5. The method of claim 1, wherein the treatment includes reducing the subject's hepatic triglyceride levels and/or collagen content.

6. The method of claim 1, wherein the pharmaceutical composition decreases LTBP3 expression, thereby decreasing LTBP3 in the liver cells and/or the pharmaceutical composition inhibits interactions of LTBP3 and pro-TGFβ homodimers, thereby decreasing LTBP3 in the liver cells.

7. (canceled)

8. The method of claim 1, wherein the fatty liver disease is nonalcoholic fatty liver disease or nonalcoholic steatohepatitis.

9. The method of claim 1, wherein the pharmaceutical composition comprises anti-LTBP antibodies.

10. The method of claim 1, wherein the pharmaceutical composition is targeted to the liver of the subject.

11. The method of claim 3, wherein administration of the LTBP3 inhibitor inhibits liver LTBP3 without significantly inhibiting LTBP elsewhere in the subject.

12. The method of claim 3, wherein the LTBP3 inhibitor is a small molecule inhibitor, an oligonucleotide, an adenoviral vector, or a CRISPR/Cas9 system for inhibiting LTBP3.

13. (canceled)

14. The method of claim 12, wherein the LTBP3 inhibitor is an oligonucleotide and wherein the oligonucleotide is an antisense oligonucleotide, an RNA-interference inducing compound, or a ribozyme.

15. The method of claim 12, wherein the oligonucleotide is targeted to hepatocytes and/or wherein the oligonucleotide is modified to increase its stability in vivo.

16. (canceled)

17. The method of claim 11, wherein the LTBP3 inhibitor is a small molecule inhibitor, an adenoviral vector, or a CRISPR/Cas9 system for inhibiting LTBP3.

18. (canceled)

19. The method of claim 18, wherein the LTBP3 inhibitor is an adenoviral vector and wherein the adenoviral vector is adenoviral ShRNA.

20. The method of claim 1, wherein the pharmaceutical composition is administered in combination with Notch-active therapies.

21. The method of claim 20, wherein the pharmaceutical composition is a LTBP3 inhibitor.

22. The method of claim 21, wherein the LTBP3 inhibitor is a small molecule inhibitor, an oligonucleotide, an adenoviral vector, or a CRISPR/Cas9 system for inhibiting LTBP3.

23-24. (canceled)

25. The method of claim 20, wherein the Notch-active therapy comprises a Notch1 decoy protein and/or comprises administering to the subject a Jagged inhibitor.

26. The method of claim 25, wherein the Notch1 decoy protein comprises (a) amino acids, the sequence of which is identical to the sequence of a portion of the extracellular domain of a human Notch1 receptor protein and (b) amino acids, the sequence of which is identical to the sequence of an Fc portion of an antibody.

27. (canceled)

28. The method of claim 25, wherein the Notch-active therapy comprises administering to the subject a Jagged inhibitor and wherein the Jagged inhibitor is a small interfering RNA for JAG1 and/or a CRISPR/Cas9 system for inhibiting JAG1.

29-30. (canceled)
